(19) United States
(12) Patent Application Publication
(10) Pub. No.: US 2025/0120609 A1
(43) Pub. Date: Apr. 17, 2025

(54) RESPIRATION DATA ANALYSIS VIA BREATH SOUNDS

(71) Applicant: Jeffrey Phillips, Pensacola, FL (US)

(72) Inventor: Jeffrey Phillips, Pensacola, FL (US)

(21) Appl. No.: 18/914,660

(22) Filed: Oct. 14, 2024

Related U.S. Application Data
(60) Provisional application No. 63/543,949, filed on Oct. 13, 2023.

(51) Int. Cl. A61B 5/08; A61B 5/00; A61B 7/00; A61F 5/08; A62B 18/02

(52) U.S. Cl. CPC A61B 5/0803; A61B 5/0022; A61B 5/0816; A61B 5/746; A61B 7/003; A61F 5/08; A62B 18/02

(57) ABSTRACT

A system for measuring and characterizing respiration-related parameters using sound. A microphone in an existing application (such as an aviation oxygen mask) can be used. Alternatively, a microphone can be added to carry out the present invention. Such a microphone is preferably added to an existing device—such as a nasal dilator.

### CROSS-REFERENCES TO RELATED APPLICATIONS

This non-provisional patent claims the benefit of previously-filed Pat. App. No. 63/543,949. The parent application was filed on Oct. 13, 2023. It listed the same inventor.

### STATEMENT REGARDING FEDERALLY SPONSORED RESEARCH OR DEVELOPMENT

Not applicable.

### MICROFICHE APPENDIX

Not Applicable

### BACKGROUND OF THE INVENTION

### 1. Field of the Invention

The present invention pertains to the field of human health. More specifically, the invention comprises a system and method for measuring human respiratory characteristics and using the data obtained to monitor human health and life support system performance.

### 2. Description of the Related Art

Respiration trends represent the most useful and accurate prediction of patient deterioration available and can identify urgent medical needs hours or days in advanced of other commonly used vital signs. An increasing respiration rate indicates distress often before the patient is even aware of the distress. Unfortunately, respiration trends are barely used in medicine today because the methods used to capture them have been tragically flawed. The standard practice is to have a caregiver visually count breaths for 15 seconds and multiply by 4 each time they do their rounds. In many cases caregivers don't count at all and simply write 18, the universal average, each time for every patient. Because the existing methods for gathering respiration data are cumbersome, this potentially valuable metric is simply not used to predict health outcomes or medical needs. Medical providers know that the standard number (19) is generally entered into the charting data and it is therefore not trusted as an actual measure of the state of the patient.

New methods to track respiration via optically monitoring changes in blood volume in the microvascular bed of tissue have been developed (photoplethysmography). However, these methods are often disrupted by patient motion and limited to a determination of respiration rate.

It would be advantageous to monitor multiple respiration characteristics using only breath sounds. The present invention takes this approach.

### BRIEF SUMMARY OF THE INVENTION

The present inventive measures and characterizes respiration-related variables using sound. A microphone in an existing application (such as an aviation oxygen mask) can be used. Alternatively, a microphone can be added to carry out the present invention. Such a microphone is preferably added to an existing device—such as a nasal dilator.

### BRIEF DESCRIPTION OF THE SEVERAL VIEWS OF THE DRAWINGS

FIG. 1 is a perspective view, showing an implementation of the present invention using a microphone in an aviation mask.

FIG. 2 is a side elevation view, showing an implementation of the present invention using a microphone added to a nasal dilator.

FIG. 3 is a block diagram, showing a representative embodiment of the present invention.

### REFERENCE NUMERALS IN THE DRAWING VIEWS

10 mask
12 gas supply
14 microphone
16 cord
18 jack
20 patient
22 nasal dilator
24 microphone
26 R/F link
28 communication module

### DETAILED DESCRIPTION OF THE INVENTION

The present invention provides the world with a simple intuitive solution to measure and characterize respiration related variables over long periods of time across a broad range of real-world applications. In situations where there is a life support system in place (tactical aviation, SCUBA, Space, HAZMAT, or Fire) the solution can be implemented by placing a tiny microphone in the oral-nasal mask or nasal cannula. If the life support system has built in communications, the existing microphone can be used. The invention provides a reliable way to track respiration that does not require any significant modifications to the life support system and therefor avoids any change that may compromise performance or safety. In other words, it doesn't require one to poke any additional holes into the life support system.

When no mask is in use a microphone and R/F communication unit (such as BLUETOOTH communication) can be built into a nasal dilation device, commonly used to stop or reduce snoring or provide increased respiratory flow. This wearable assembly is capable of capturing valid real-time respiration trends during exercise, work, sleep, or relaxation/meditation.

The invention will preferably be paired with a software application that runs on PC, Mac, IOS and Android that provides respiration trend feedback across a range of activity modes from meditation and relaxation to extreme physical exertion. The application will include a respiration coach that emphasizes nasal over oral breathing and deep over shallow breathing.

When used in conjunction with medical devices or advanced life support systems, the invention will be capable of learning to detect life support system malfunctions and diagnose issues when and wherever a system is in use through audio feature analysis methodology. This will prevent catastrophic failures by ensuring proper maintenance and optimal performance in between maintenance and certification cycles. The invention will also save stakeholders millions by pinpointing the problem to subcomponents of a larger system. For example, a malfunctioning breathing regulator can easily be identified without having to dismantle and troubleshoot the entire system. This is extremely important in aviation, undersea and space operations.

The invention has the capacity to revolutionize the way respiration is measured and modeled across all medical, professional, and personal markets. It will provide the best method to monitor vital signs since the invention of pulse oximetry and electrocardiogram. The implementation of the invention would have a vast positive effect on medical outcomes and general health and wellness.

Embodiments of the invention will preferably have the ability to determine (1) respiration rate, (2) inhalation time, (3) exhalation time, (4) Exhalation volume, (5) Inhalation volume, (6) inhalation volume, (7) exhalation volume, (8) pauses between breaths or cycles, (9) minute-by-minute respiration rate, and (10) minute ventilation.

More advanced features will include AI that learns your personal respiration trends across a range of activity levels to detect off-nominal states. The AI will also serve as a respiration coach that guides respiration through prompts and provides direct biofeedback to track and improve performance.

The microphone transforms sound into electrical signals and the auditory processor in the present invention uses these signals to monitor respiration. The most useful signal is a spike associated with the start of inhalation. This can be counted as the start of one respiration cycle, so that the number of spikes detected in a fixed interval of time can be used to calculate a respiration rate.

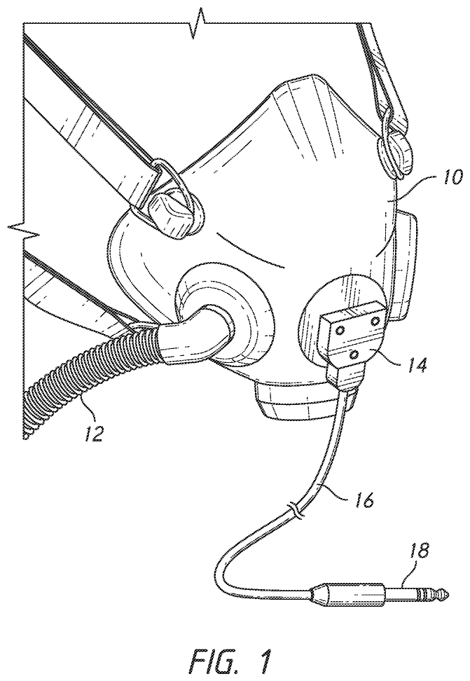

The invention can be physically implemented in a wide variety of ways. FIGS. 1-3 show some exemplary embodiments. FIG. 1 is a perspective view of an aviation mask 10. Gas supply 12 is fed into one side of the mask. Microphone 14 is a built-in microphone that is used for communications. It attaches to jack 18 via cord 16. This existing microphone can be used to capture respiration sounds that the inventive system employs in its analysis.

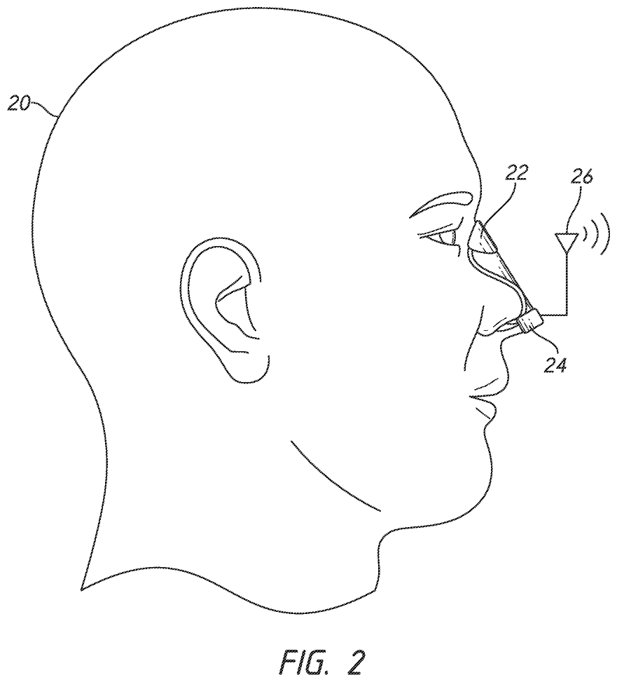

FIG. 2 illustrated a scenario where no microphone is ordinarily present. Nasal dilator 22 is anchored across the bridge of the nose and its two pliable legs are inserted into the nostrils. The pliable leg for the right nostril is the thin curved structure shown in the view. As will be known to those skilled in the art, such devices aid nasal respiration by opening the nostrils and are helpful in most situations. A small microphone 24 can be added to the nasal dilator in a position suitable for capturing respiration sounds.

It is possible to attach a cord to microphone 24, but this tends to limit patient motion and may also be uncomfortable. A better solution is to provide a power supply on the device (preferably a rechargeable one) and an R/F link 26 that can be used to connect to one or more external monitoring devices. The R/F link preferably uses a standardized communication protocol such as the BLUETOOTH protocol (managed by the Bluetooth Special Interest Group of Kirkland, Washington, U.S.A.) or the ZIGBEE protocol (managed by the Connectivity Standards Alliance of Davis, California, U.S.A.). The R/F link connects the microphone to an external data collection and analysis system. It is also possible to do some processing on the device and only transmit parameters determined locally (such as a transmission at the onset of each detected inhalation).

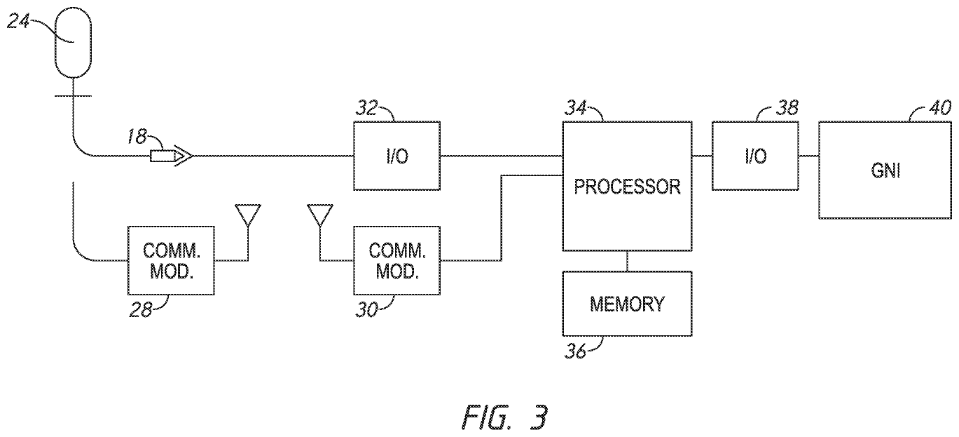

FIG. 3 is a block diagram depicting exemplary hardware and software. Microphone 24 can be connected to processor 34 via a hard line and I/O module 32 (as in the example of mask 10 in FIG. 1). Alternatively, the microphone can be connected to the processor via two communications modules 28,30 and an R/F link established between the two (as in the example of nasal dilator 10 in FIG. 2).

Processor 34 hosts the data analysis software. Data and processing results are stored in associated memory 36. A separate I/O module 38 connects the processor to a graphical user interface 40. The GUI allows a user to conveniently interact with the system. Those skilled in the art will appreciate that most of the components shown in FIG. 3 can be manifested in a single device—such as a smartphone or micro-computer. Thus, the user interface may be a simple and dedicated device—such as the display of a computed respiration rate on a patient monitor that also displays heart rate, blood pressure, and blood oxygen levels. The user interface may also be an “app” running on a smartphone.

Some embodiments will include a much simpler user interface. As an example, the user interface might be an “alarm” function that provides an alert on an existing patient monitoring system when the patient's respiration rate has increased pas a defined threshold, or has exceeded a defined rate of change. Once the alarm is provided, the healthcare provider could be given the option to pull up the actual respiration data and look at the respiration rate over time.

The preceding description contains significant detail regarding the novel aspects of the present invention. It should not be construed, however, as limiting the scope of the invention but rather as providing illustrations of the preferred embodiments of the invention. Thus, the scope of the invention should be fixed by the claims ultimately drafted, rather than by the examples given.

Having described my invention, I claim:

1. A device for monitoring a respiration rate in a patient, comprising:
(a) a nasal dilator;
(b) a microphone mounted on said nasal dilator;
(c) a first communication module mounted on said nasal dilator and connected to said microphone;
(d) a second communication module mounted remote from said nasal dilator;
(e) a processor connected to said second communication module;
(f) a display connected to said processor;
(g) said first communication module being configured to receive electrical signals from said microphone and transmit data signals based on said electrical signals to said second communication module;
(h) said processor being configured to use said data signals from said second communication module to determine a respiration rate for said patient; and
(i) said display being configured to display said respiration rate determined by said processor.

2. The device for monitoring a respiration rate as recited in claim 1, further comprising a rechargeable power source mounted on said nasal dilator, said power source providing electrical power to said microphone and said first communication module.

3. The device for monitoring a respiration rate as recited in claim 1 wherein said first and second communication modules communicate using radio signals.

4. The device for monitoring a respiration rate as recited in claim 1, wherein said processor produces an alarm signal when said respiration rate increases beyond a defined threshold.

5. The device for monitoring a respiration rate as recited in claim 3 wherein said radio signals are sent using a BLUETOOTH format.

6. The device for monitoring a respiration rate as recited in claim 3 wherein said radio signals are sent using a ZIGBEE format.

7. The device for monitoring a respiration rate as recited in claim 1, wherein said first and second communication modules are connected by a wire.

8. A device for monitoring respiration in a patient, comprising:
(a) a nasal dilator;
(b) a microphone mounted on said nasal dilator, said microphone being configured to receive sound information corresponding to said respiration of said patient and convert said sound information into electrical signals;
(c) a first communication module mounted on said nasal dilator and connected to said microphone;
(d) a second communication module mounted remote from said nasal dilator;
(e) a processor connected to said second communication module;
(f) a display connected to said processor;
(g) said first communication module being configured to receive said electrical signals from said microphone and transmit data signals based on said electrical signals to said second communication module;
(h) said processor being configured to use said data signals from said second communication module to determine at least one respiration parameter for said patient; and
(i) said display being configured to display said respiration parameter determined by said processor.

9. The device for monitoring a respiration rate as recited in claim 8, further comprising a rechargeable power source mounted on said nasal dilator, said power source providing electrical power to said microphone and said first communication module.

10. The device for monitoring a respiration rate as recited in claim 8 wherein said first and second communication modules communicate using radio signals.

11. The device for monitoring a respiration rate as recited in claim 8, wherein said processor produces an alarm signal when said respiration rate increases beyond a defined threshold.

12. The device for monitoring a respiration rate as recited in claim 10 wherein said radio signals are sent using a BLUETOOTH format.

13. The device for monitoring a respiration rate as recited in claim 10 wherein said radio signals are sent using a ZIGBEE format.

14. The device for monitoring a respiration rate as recited in claim 8, wherein said first and second communication modules are connected by a wire.
